(19) United States
(12) Patent Application Publication
(10) Pub. No.: US 2002/0028849 A1
(43) Pub. Date: Mar. 7, 2002

(54) Use of retinol in assisted-reproduction protocols

(76) Inventors: James D. Godkin, Knoxville, TN (US); Dawn M. Eberhardt, Knoxville, TN (US)

Correspondence Address: Stanley A. Kim, Akerman, Senterfitt & Eidson, P.A., 222 Lakeview Avenue, Suite 400, P.O. Box 3188, West Palm Beach, FL, 33402-3188, US

(21) Appl. No.: 09/837,156

(22) Filed: Apr. 17, 2001

Related U.S. Application Data
(60) Provisional application No. 60/198,061, filed on Apr. 18, 2000.

(51) Int. Cl. A61K 31/203; A61K 31/07; C12N 15/00

(52) U.S. Cl. 514/560; 800/21; 514/725

(57) ABSTRACT

Disclosed is the use of retinoids such as retinol, all trans retinoic acid, and 9-cis retinoic acid to enhance the success of assisted-reproduction. Administration of retinol to superovulated animals dramatically improved embryo viability and development as well as the pregnancy rates of animals implanted with embryos derived from such animals. Culturing presumptive embryos in vitro in the presence of retinol enhanced development of embryos from the presumptive zygotes compared to presumptive embryos not treated with retinol.

### CROSS REFERENCE TO RELATED APPLICATION

The present application claims the benefit of U.S. provisional patent application number 60/198,061 filed Apr. 18, 2000.

### STATEMENT AS TO FEDERALLY SPONSORED RESEARCH

Not applicable.

### FIELD OF THE INVENTION

The invention relates the field of sexual reproduction. More particularly, the invention relates to the use of retinoids to improve assisted-reproduction protocols.

### BACKGROUND OF THE INVENTION

Reproductive failure is a serious problem that has been addressed clinically by induced superovulation (e.g., administering gonadotropin to a female to induce the ovaries to produce more than a typical number of ova), in vitro fertilization (IVF) and embryo transfer (ET). Each of these procedures can assist in achieving high conception rates as, for example, superovulation provides many ova that might be fertilized and IVF/ET allows numerous fertilized ova to be implanted into a primed recipient. Despite these efforts the success rate of such assisted-reproductive techniques remains less than ideal.

Retinol (Vitamin A; e.g., all-trans retinol (ROH)) and its cellular metabolites, all-trans retinoic acid (RA), 9-cis retinoic acid (CIS), and derivatives of the foregoing are collectively known as retinoids. These compounds influence embryonic morphogenesis, cell growth, and differentiation in many cell types including embryonic stem cells and embryo carcinoma cells. Differentiation induced by retinoids in vitro is accompanied by specific changes in expression of homeobox genes, growth factors, and their receptors. Gudas et al., Cellular Biology and Biochemistry of the Retinoids. In: Sporn MB, Roberts AB, Goodman DS (eds.), The Retinoids-Biology, Chemistry and Medicine. New York: Raven Press, Ltd; 1994: 443-520. Additionally, retinoids have been shown to play an important role in reproduction in both males and females, as for example, deficiencies in vitamin A lead to decreased ovarian size, decreased ovarian steroid concentrations, abortion, and eventually reproductive senescence. Mangelsdorf et al., Cellular Biology and Biochemistry of the Retinoids. In: Sporn MB, Roberts AB, Goodman DS (eds.), The Retinoids-Biology, Chemistry and Medicine. New York: Raven Press, Ltd; 1994: 319-350.

### SUMMARY OF THE INVENTION

The invention is based on the discovery that administration of retinol to superovulated animals dramatically improves embryo viability and development. It was also discovered that culturing presumptive zygotes (i.e., oocytes collected from ovaries, matured in vitro for 24 h, and then fertilized using standard IVF procedures) in vitro in the presence of retinol enhanced development of embryos from the presumptive zygotes. Thus the invention relates to the use of retinoids to enhance reproductive success in animals.

Accordingly, the invention features a method for enhancing reproductive success in an animal. This includes the step of administering to the animal (a) a preparation including a retinoid in an amount effective to enhance the reproductive success of the animal and (b) an agent that stimulates superovulation in an amount sufficient to stimulate superovulation in the animal. The preparation used in this method can further include a pharmaceutically acceptable carrier. The step of administering the preparation can be performed by a parenteral route, e.g., by injection. The retinoid used in this method can be all-trans retinol. All-trans retinol can be administered to the animal in a dosage of 500 to 50,000 IU/Kg, e.g. 1000 to 25,000 IU/Kg.

In another aspect the invention features a method for enhancing the viability of an embryo. In first variation, this method includes the steps of: (a) isolating an ovum from an animal; (b) fertilizing the isolated ovum to form an embryo; and (c) exposing the embryo to a purified retinoid.(e.g., retinol or retinoic acid). In this variation, the embryo can be exposed to the purified retinoid at a concentration of 0.05 to 50 micromolar (e.g., 1 to 10 micromolar) and for a period of at least one hour (e.g. 24 hours). A second variation of this method includes the steps of: (a) isolating an ovum from an animal;(b) exposing the isolated ovum to a purified retinoid; and; (c) fertilizing the ovum to form an embryo. In the second variation, the ovum can be exposed to the purified retinoid at a concentration 0.05 to 50 micromolar (e.g., 1 to 10 micromolar) and for a period of at least one hour (e.g. 24 hours). Both variations of this method can include an additional step (d) of implanting the embryo in a uterus or a fallopian tube.

Also within the invention is an embryo made according to a process that includes the steps of: (a) isolating an ovum from an animal; (b) fertilizing the isolated ovum to form an embryo; and (c) exposing the embryo to a purified retinoid (e.g., retinol or retinoic acid). An embryo made according to a process that includes the steps of: (a) isolating an ovum from an animal; (b) exposing the isolated ovum to a retinoid; and (c) fertilizing the ovum to form an embryo is likewise within the invention.

In still another aspect the invention features a kit for enhancing reproductive success in an animal. The kit includes at least one dose of a purified retinoid, and written instructions for administering the dose to the animal. The at least one dose includes a sufficient amount of the retinoid to enhance the reproductive success of the animal after being administered to the animal. In the kit, the retinoid can be retinol, and the at least one dose of a retinoid can be formulated for parenteral administration. The retinoid within the kit can be mixed with a pharmaceutically acceptable carrier.

As used herein, the term “purified” means separated from components that naturally accompany the thing that has been purified. For example, a retinoid is purified when it is a least 10% (e.g., 20, 30, 40, 50, 60, 70, 80, 90, 95, 96, 97, 98, 99 or more percent) free from other molecules that naturally accompany it. Purity can be measured by conventional methods such as HPLC. A purified substance can also be (a) one made synthetically or (b) one that has been isolated from a natural source (e.g., an animal), mixed with at least one other substance not originating from the natural source to form a mixture, and then added back to the natural source in the mixture.

By the phrase “specifically binds” is meant that one molecule in a mixture recognizes and adheres to a particular second molecule in the mixture, but does not substantially recognize or adhere to other molecules (dissimilar from the second molecule) in the mixture.

By “retinoid” is meant any substance that can specifically bind a retinoid binding protein such as ROH, RA, CIS, and naturally occurring and synthetic derivatives of the foregoing. Examples of retinoids include retinols, retinoic acids, and retinyl esters. By the term “retinol” is meant any isomers of retinol, e.g., all-trans-retinol, 13-cis-retinol, 11-cis-retinol, 9-cis-retinol, 3,4-didehydro-retinol. All-trans-retinol is preferred for some aspects of the invention, due to its wide commercial availability. Examples of retinoic acids include all trans retinoic acid (tretinoin) and 9-cis retinoic acid. Examples of retinyl esters include: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, and retinyl oleate. Other synthetically prepared retinoids might be used in the invention, see e.g., U.S. Pat. Nos. 4,326,055 and 5,234,926.

As used herein, the phrase “enhancing the reproductive success of an animal” means increasing the likelihood that animal will bear offspring, e.g., by increasing the number of ova produced by the animal, increasing the likelihood that one of the animal's ova will become fertilized, or increasing the ova's ability of to become fertilized, or the chance that a fertilized ova (i.e., zygote or embryo) will develop into another animal.

By the phrase “enhancing the viability of an embryo” is meant increasing the likelihood that the embryo will progress in the developmental cycle (e.g., increase in cell number and/or continue to differentiate).

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including any definitions will control. In addition, the particular embodiments discussed below are illustrative only and not intended to be limiting.

### BRIEF DESCRIPTION OF THE DRAWINGS

The above and further advantages of this invention may be better understood by referring to the following description taken in conjunction with the accompanying drawings, in which:

FIG. 1A is a graph showing that embryos from the ROH treated ewes had a greater than 2-fold increase in vitro blastocyst formation compared to RA-treated, CIS-treated, or Control animals (72% vs. 27%, 33%, and 32%; p<0.05).

FIG. 1B is a graph showing that ROH treating superovulated ewes improved (p<0.05) embryonic hatching rates in vitro in comparison with the rates for CIS and Control animals but was not different from that for RA-treated animals (73%, 38%, 36%, and 55%, respectively).

FIG. 1C is a graph showing that treating donor ewes with ROH resulted in a dramatic increase in the percentage of embryos that formed blastocysts compared with the control value (70% vs. 22%; n=243 and 218, respectively).

FIG. 1D is a graph showing that ROH treatment of superovulated ewes resulted in an increase to nearly 3-fold in hatching rate in comparison with vehicle treatment (70% vs. 27%, p<0.05).

FIG. 2 is a graph showing that ROH treatment of superovulated ewes significantly (p<0.05) improved the number of embryos that progressed through the 8-cell in vitro block (94% vs. 40%), and resulted in a dramatic increase (p<0.05) in blastocyst formation (79% vs. 5%; n=230 and 202, respectively) and blastocyst hatching (71% vs. 0%, respectively).

### DETAILED DESCRIPTION OF THE INVENTION

A series of experiments was conducted to identify effects of retinoid treatment of superovulated ewes upon subsequent in vitro embryonic development. In one such experiment, ewes were treated with ROH, RA, CIS, or vehicle (Control) on the first and last day of FSH treatment. Embryos were recovered at the morula stage, cultured, and observed for blastocyst formation. Embryos from ROH-treated animals had a higher incidence of blastocyst formation than RA-, CIS-, or vehicle-treated animals. In another experiment, ewes were administered ROH or vehicle and treated as above. ROH treatment resulted in an increased percentage of embryos forming blastocysts. In still another experiment, ewes were treated with ROH or vehicle, and embryos were collected at the 1- to 4-cell stage and cultured for 7 days. Using this protocol, ROH treatment resulted in a dramatic increase in blastocyst formation among embryos from ROH-treated animals compared to those from vehicle-treated animals. ROH treatment of superovulated ewes was thus shown to increase embryonic viability and positively impact embryonic development.

In another series of experiments, the effects of retinol and retinoic acid on early embryonic development of in vitro-produced bovine embryos was investigated. Oocytes and their surrounding cumulus cells were collected from bovine ovaries, matured in vitro, and fertilized by standard procedures. Presumptive zygotes were denuded of cumulus cells, washed and cultured in modified synthetic oviduct fluid (a standard culture medium) in the presence or absence of retinol. The presumptive zygotes were then cultured for several days, and blastocyst formation was analyzed as an end point for in vitro development. The results of this experiment showed that retinol significantly increased development to the blastocyst stage. Similar positive results were observed when the culture medium contained RA.

Still another set of experiments was conducted to identify the effects of retinoid treatment of superovulated animals upon the ability of embryos derived from the animals to induce pregnancy in recipient animals. In these experiments, superovulated and synchronized ewes were treated with retinoids or control, and then fertilized. Embryos were recovered from the animals, frozen, and later implanted in the uteri of recipient animals to examine the effect of the retinoid treatment on pregnancy rates. Eighty-six (86) % of the ewes receiving embryos from retinol-treated ewes were determined to be pregnant, whilst only 45% of the ewes receiving embryos from control treated ewes were observed to be pregnant. Of the six pregnant ewes carrying embryos from retinol-treated ewes, five were observed to be carrying twins and one was carrying a single embryo. All five of the pregnant ewes carrying embryos from control ewes were observed to be carrying single embryos. Of all transferred embryos from retinol-treated ewes, 78.6% survived, versus a 22.7% survival rate of embryos from control-treated ewes.

Thus the invention encompasses methods and compositions for enhancing the success of assisted-reproduction techniques. The preferred embodiments described herein illustrate various methods and compositions for enhancing the reproductive success of sheep and cattle by treating an animal or a presumptive zygote with one or more retinoids. From the description of these embodiments, other methods and compositions can be made by making slight modifications to the methods and compositions discussed below.

Materials

The materials used in the invention are commercially available form one or more sources. Sources of the materials used in the experiments described herein are as follows. Lutalyse was obtained from the Upjohn Company (Kalamazoo, Mich.). Synchromate B was obtained from Rhone Merieux, Inc. (Athens, Ga.). Prostaglandin F2-alpha (Lutalyse) was obtained from the Upjohn Company (Kalamazoo, Mich.). 9-cis retinoic acid was obtained from Hoffmann La Roche (Nutley, N.J.). Porcine FSH was obtained from Sioux Biochemical (Sioux City, Iowa). Fetal bovine serum (FBS) was obtained from Atlanta Biologicals (Norcross, Ga.). Synthetic oviductal fluid (SOF) was obtained from Specialty Media, Inc. (Lavallette, N.J.). Falcon organ culture dishes was obtained from Fisher Scientific (Pittsburgh, Pa.). All-trans retinol, all-trans retinoic acid, and all other reagents were obtained from Sigma Chemical Co. (St. Louis, Mo.).

Animals

The invention is believed to be compatible with any animal having an estrous or a menstrual cycle. A non-exhaustive list of examples of such animals includes mammals such as mice, rats, rabbits, goats, sheep, pigs, horses, cattle, dogs, cats, and primates such as monkeys, apes, and human beings. In the experiments described herein, the animals used were sheep and cattle. Nonetheless, by adapting the methods taught herein to other methods known in medicine or veterinary science (e.g., adjusting doses of administered substances according to the weight of the subject animal, administering the appropriate gonadotropin at the appropriate time to induce superovulation in the subject animal, etc.) can be readily optimized for use in other animals. See, e.g., Encyclopedia of Reproduction, E. Knobil and J. Neill (Eds.), Academic Press, San Diego, 2000.

Administration of Compositions

The compositions of the invention may be administered to animals including humans in any suitable formulation. For example, retinoids may be administered in neat form. They may also be formulated in pharmaceutically acceptable carriers or diluents such as physiological saline or a buffered salt solution. Suitable carriers and diluents can be selected on the basis of mode and route of administration and standard pharmaceutical practice. For example, retinol is practically insoluble in water or aqueous salt solutions, but soluble in ethanol, methanol, ether, fats, and oil. For injection, retinol can be dissolved in corn oil. For addition to tissue culture, retinol can be dissolved in a very small amount of ethanol. A description of other exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions. For example, glycine (e.g., 0.3M, pH 6.8), maltose (e.g., 10%) and/or thimerosal (e.g., 1:10,000) may be added to the compositions.

The compositions of the invention may be administered to animals by any conventional technique. Typically, such administration will be parenteral (e.g., intravenous, subcutaneous, intramuscular, or intraperitoneal introduction). The compositions may also be administered directly to the target site (e.g., an ovary) by, for example, surgical delivery to an internal or external target site, or by catheter to a site accessible by a blood vessel. Other methods of delivery, e.g., liposomal delivery or diffusion from a device impregnated with the composition, are known in the art. Oral delivery of the compositions might also be used in some cases. The compositions may be administered in a single bolus, multiple injections, or by continuous infusion (e.g., intravenously or by peritoneal dialysis). For parenteral administration, the compositions are preferably formulated in a sterilized pyrogen-free form.

Compositions of the invention can also be administered in vitro to isolated ova or embryos by simply adding the composition to the fluid in which an isolated ovum or embryo is contained. For example, to expose a presumptive zygote (i.e., either an ovum exposed to spermatozoa or an embryo) to retinol, the retinol is dissolved in a synthetic oviduct fluid at a suitable concentration (e.g., 0.001-100 micromolar ROH such as 0.05-50, 0.1-10, 0.5-5, 0.0008, 0.001, 0.002, 0.005, 0.01, 0.02, 0.05, 1, 2, 3, 4, 5, 6, 7, 8, 9, 10, 10, 30, 40, 50, 60, 70, 80, 90, 100 or 110 micromolar ROH), and the ovum or embryo is added to the retinol-containing synthetic oviduct fluid. As another example, an isolated oocyte can be exposed to retinol by culturing the oocyte in a maturation medium (i.e., any medium that supports maturation of an isolated oocyte in vitro; e.g., tissue culture medium 199 (TCM-199, Sigma Chemical Co., St. Louis, Mo.) containing 0.2 mM pyruvate, 5.0 ug/ml FSH, 1.0 ug/ml estradiol and 10% fetal bovine serum (Sirard MA, Parrish JJ, Ware, CB Leibfried-Rutledge, ML and First NL, Biology of Reproduction, 1988; 39: 546-552)) containing a retinoid such as retinol or retinoic acid in a concentration of about 0.001-100 micromolar (e.g., 0.05-50, 0.1-10, 0.5-5, 0.0008, 0.001, 0.002, 0.005, 0.01, 0.02, 0.05, 1, 2, 3, 4, 5, 6, 7, 8, 9, 10, 10, 30, 40, 50, 60, 70, 80, 90, 100 or 110 micromolar).

Effective Doses

An effective amount is an amount which is capable of producing a desirable result in a treated animal (e.g., enhanced reproductive ability) or cultured cell or embryo (e.g., enhanced viability). As is well known in the medical and veterinary arts, dosage for any one animal depends on many factors, including the particular animal's size, body surface area, age, the particular composition to be administered, time and route of administration, general health, and other drugs being administered concurrently. It is expected that an appropriate retinoid dosage for intramuscular administration of retinoids would be in the range of about 0.001 to 100 mg/kg (e.g., 0.1-10, 10-100, 20-50, 0.001, 0.005, 0.01, 0.05, 1, 2, 3, 4, 5, 10, 20, 30, 40, 50, 60, 60, 80, 90, 100, or 110 mg/kg) body weight. For example, 500 to 50,000 IU/kg (more particularly, 1000 to 25,000 IU/kg) is an effective dose from many animals, but this the actual dose that is effective may vary depending on the species. An effective amount for use with a cultured ovum or embryo will also vary, but can be readily determined empirically (e.g., by adding varying concentration to the ovum or embryo and selecting the concentration that best produces the desired result). It is expected that an appropriate concentration would be in the range of about 0.001-100.0 micromolar (e.g., 0.01-50, 0.1-10, 0.5-5, 0.0008, 0.001, 0.002, 0.005, 0.01, 0.02, 0.05, 1, 2, 3, 4, 5, 6, 7, 8, 9, 10, 10, 30, 40, 50, 60, 70, 80, 90, 100 or 110 micromolar) for use with a cultured ovum or an embryo. For example, exposure to 0.05 to 50 micromolar (more particularly, 0.1 to 10.0 micromolar) for greater than about 1 h (e.g., 0.8 h, 1 h, 1.5 h, 2 h, 3 h, 6 h, 12 h, 24 h or more) is an effective dose from many animal embryos or ova, but this the actual dose that is effective may vary depending on the species. More specific dosages can be determined by the method described below.

Toxicity and efficacy of the compositions of the invention can be determined by standard pharmaceutical procedures, using cells in culture, ova, embryos, and/or experimental animals to determine the LD50 (the dose lethal to 50% of the population) and the ED50 (the dose that effects the desired result in 50% of the population). Compositions that exhibit a large LD50/ED50 ratio are preferred. Although less toxic compositions are generally preferred, more toxic compositions may sometimes be used in in vivo applications if appropriate steps are taken to minimize the toxic side effects.

Data obtained from cell culture and animal studies can be used in estimating an appropriate dose range for use in humans. A preferred dosage range is one that results in circulating concentrations of the composition that cause little or no toxicity. The dosage may vary within this range depending on the form of the composition employed and the method of administration.

### EXAMPLE 1

### Retinoid Administration to Superovulated Animals

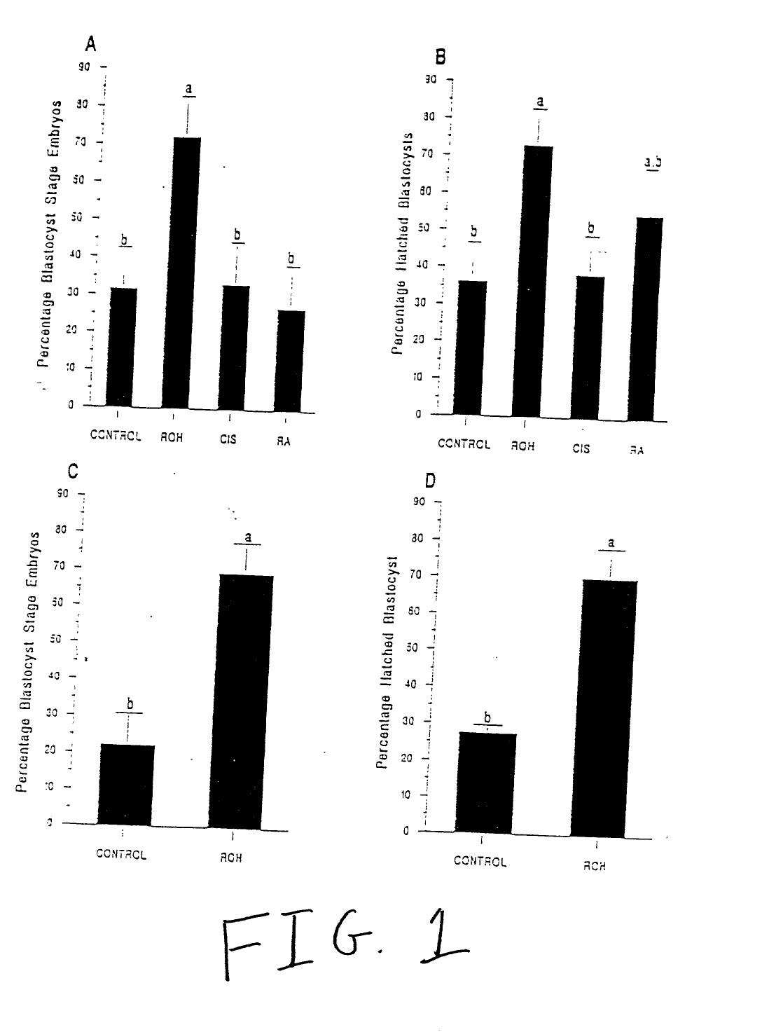

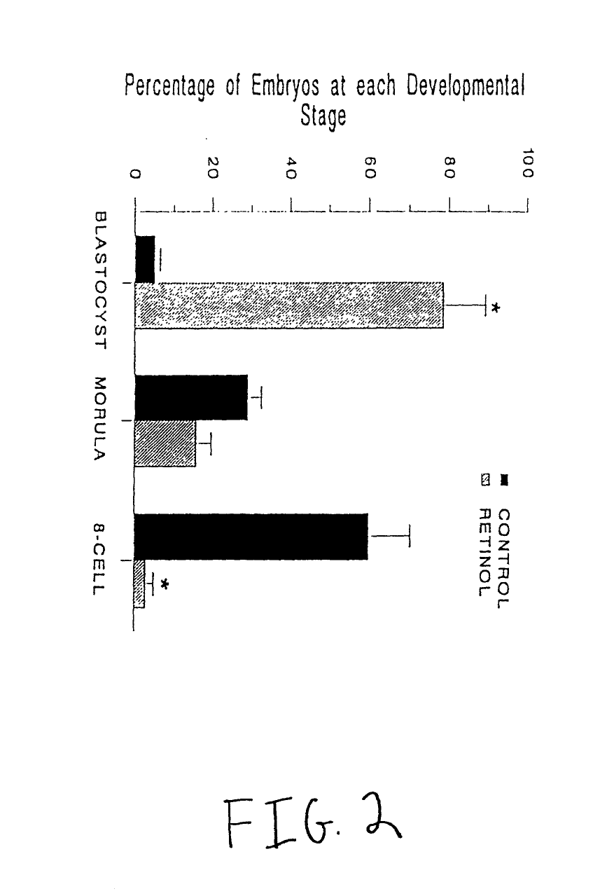

Referring now to FIGS. 1 and 2, a series of experiments were carried out to examine the effect of retinoid administration on superovulated sheep. In these experiments, the estrous cycles of sexually mature crossbred ewes were synchronized using progestin implants (Synchromate B) combined with prostaglandin F2-alpha (Lutalyse) injections, and superovulation was induced by multiple FSH injections. Briefly, animals were administered one progestin implant and 6 days later received two Lutalyse injections (15 mg i.m.) 12 hours apart. Superovulation was induced using a total of 24 units of FSH administered twice daily in decreasing doses over 3 days (5.5, 4.4, 3.3 units per injection, respectively) beginning 9-11 days after implant administration. Retinoid treatments were administered on the first and last day of FSH injections. Implants were removed at the time of the fifth FSH injection, and animals were checked for estrus 24 hours later. Ewes exhibiting behavioral estrus were hand-bred to intact rams every 12 hours until signs of estrus were no longer detected. All animals were maintained on high-quality hay and fed ad libitum, with free-choice access to a sheep and goat mineral premix that contained 1 million IU vitamin A per pound.

In the first experiment, performed under decreasing day length (fall), 25 ewes were randomly assigned to one of the following treatments: (1) all-trans retinol (ROH; 500,000 IU, n=6); (2) all-trans retinoic acid (RA; 15 mg, n=6); (3) 9-cis retinoic acid (CIS; 15 mg, n=7); or (4) vehicle (Control; n=6), which was corn oil. Animals were surgically ovohysterectomized at 144 hours post-implant removal, and uteri were gently flushed twice with culture medium (tissue culture medium 199 [TCM 199]) to collect morula stage embryos. Two ewes from the RA group wer dropped from the study: one because of a total lack of response to the FSH treatment, the other because of overstimulation resulting in more than 50 ovulations with none of the ova fertilizing. This left 4 animals in the RA group, 5 in each of the ROH and Control groups, and 7 in the CIS group.

The second experiment was a repetition of the first, with the exceptions that it was performed under increasing day length (winter) and only the ROH and Control treatments were administered (in combination with FSH) to 24 ewes (12 per treatment) not used in the previous experiment. One ewe from the Control group was dropped from the study for failure to respond to FSH treatment.

The third study involved two identical experiments performed sequentially in the fall and winter. Results were not different between seasons, and the data were combined. A total of 24 ewes, not used in the previous experiments, received either ROH (n=12) or Control (n=12) treatment in combination with FSH, followed by natural mating at estrus, as in experiment 2. At 84 hours post-implant removal, ewes were salphincectomized and oviducts gently flushed with culture medium in order to recover 1- to 4-cell embryos.

At the time of embryo recovery, ovulation rate was determined by counting corpora lutea (CL) on each ovary. Embryo/oocyte recovery rates were determined by dividing the embryo/oocyte number by CL number. Fertilization rate was determined by dividing the number of cleaved embryos by the total number of embryos/oocytes recovered from each ewe.

Embryos (morulae) collected in experiments 1 and 2 were categorized according to morphology, developmental stage, and quality based on a procedure developed for bovine embryos. See, Lindner and Wright, Theriogenology 1983; 20:407-416. Quality grades ranged from 1 to 4, with 1=excellent, 2=good, 3=poor, and 4=degenerate. One individual, who was unaware of treatments at the time, performed all of the grading.

In the first two experiments, morula-stage embryos were cultured in TCM 199 with Earle's salt supplemented with 10% FBS and 1 mM glutamine. See, Bavister BD. Studies on the developmental blocks in cultured hamster embryos. In: Bavister BD (ed.), The Mammalian Preimplantation Embryo: Regulation of Growth and Differentiation in vitro. New York:Plenum Press; 1987:219-249. The FBS had been twice stripped of low molecular weight molecules with charcoal, and retinol concentrations were below detection levels as determined by fluorescent analysis. See, Selvaraj and Susheela, Clin Chim Acta 1970; 27:165-170. In the third experiment, 1- to 4-cell embryos were cultured in SOF supplemented with 3 mg/ml BSA and essential and nonessential amino acids. See, Carolan et al., Theriogenology 1995; 43:1115-1128. Both media were prepared weekly, filtered through a 0.2 um filter, and allowed to equilibrate for 2 hours in a humidified atmosphere at 38.5° C. containing 5% CO₂ in air.

Morula-stage embryos (experiments 1 and 2) were washed a minimum of three times in the outer well of organ culture dishes and then transferred with a minimum amount of medium into the inner well, which contained 3 ml of TCM 199. Embryos from each ewe were cultured in one dish, and there were no significant differences in the average number of embryos per dish between treatments. Embryos were cultured for 96 hours and observed daily for blastocyst formation and complete hatching from the zona pellucida. No further development was observed after 72 hours in culture, and all data presented reflect that time period.

In experiment 3, embryos were treated as above except that culture medium was SOF (see above). Embryos were observed every 48 hours until embryos hatched or failed to develop for two consecutive viewings (168-h maximum).

Data were checked for normality and analyzed using the Statistical Analysis System (SAS Institute Inc., Cary, N.C.). ANOVA was used with mixed models procedure (PROC MIXED) to detect differences in ovulation rate, embryo recovery rate, fertilization rate, embryonic quality, in vitro embryonic development to blastocyst, and embryonic hatching due to retinoid treatment. Differences due to retinoid treatment were tested utilizing protected least-significant difference.

Ovulation rate was not affected by retinoid treatment within any experiment or between experiments, and ranged from 8 to 33, with an average of 19.33 CL per ewe excluding data from the animal that did not respond to FSH treatment and the one animal from which>50 unfertilized oocytes were recovered. Embryo/oocyte recovery rates were not different between treatments (p<0.50) or experiments (p<0.32) and ranged from 82% to 93%. Fertilization rate was also not influenced by retinoid treatment (p<0.12) and ranged from 83% to 93%. No differences were observed in in vivo developmental stage at time of collection or in speed of development (progression to the next developmental stage) in vitro. Results from experiments performed during decreasing day length (fall) were not different from those for experiments performed during increasing day length (winter).

Retinol in combination with superovulation significantly improved embryonic viability as measured by blastocyst formation in vitro. In the first experiment, embryos were collected from 21 ewes treated with ROH (n=5), RA (n=4), CIS (n=7), or Control (n=5), resulting in 96, 84, 97, and 93 embryos per group, respectively. Embryos from each treatment were graded immediately after collection, and the score was not different between treatments (1.9±0.1, 2.8-0.2, 2.4±0.2, and 2.1±0.1 for ROH, RA, CIS, and Control, respectively). Embryos from the ROH-treated animals had a greater than 2-fold increase in vitro blastocyst formation than those from RA, CIS, or Control animals (72% vs. 27%, 33%, and 32%; p<0.05) (FIG. 1A). In addition, ROH treatment improved (p<0.05) embryonic hatching rates in vitro in comparison with the rates for CIS and Control animals but was not different from that for RA-treated animals (73%, 38%, 36%, and 55%, respectively) (FIG. 1B).

In the second experiment, treatment of donors with ROH resulted in a dramatic increase in the percentage of embryos that formed blastocysts compared with the control value (70% vs. 22%; n=243 and 218, respectively) (FIG. 1C). ROH treatment resulted in an increase to nearly 3-fold in hatching rate in comparison with vehicle treatment (70% vs. 27%, p<0.05) (FIG. 1D).

In the third experiment (FIG. 2), the effect of ROH treatment of the dam (24 ewes) on in vitro development of 1 to 4-cell embryos was investigated. ROH treatment significantly (p<0.05) improved the number of embryos that progressed through the 8-cell in vitro block (94% vs. 40%). As in the first two experiments, ROH treatment resulted in a dramatic increase (p<0.05) in blastocyst formation (79% vs. 5%; n=230 and 202, respectively) and blastocyst hatching (71% vs. 0%, respectively).

Experiments were performed over a period of 2 years under conditions of both decreasing (fall) and increasing (winter) day length and included over 70 ewes producing more than 1300 embryos. Results from every experiment demonstrated that ROH treatment, in combination with superovulation, dramatically improved the in vitro developmental competence of resultant embryos. In the first experiment, the incidence of blastocyst formation and hatching of embryos from animals treated with ROH, but not RA, was dramatically higher than for embryos from vehicle-treated animals.

In the above-described experiments, while an increase in embryonic viability in vitro was observed, no influence of retinol treatment of ewes on the quality, as judged by embryo score, or quantity of morula collected. Retinoid treatment also did not affect the rate (speed) of in vivo development. In vitro development of embryos from control and retinol treated ewes was parallel, in terms of time, up until the time when controls failed to progress. To prevent exposing embryos to retinol-containing medium in vitro, the serum component of the medium used was twice stripped using a charcoal treatment that removed all detectable retinol. Embryos were cultured in 3 ml of medium in organ culture dishes to minimize evaporation and temperature change during handling. In other experiments, using immunolocaliztion, the binding proteins for retinol (RBP and CRBP) were found in the thecal cells of healthy but not atretic antral follicles in the ewe.

### EXAMPLE 2

### In Vitro Retinol Treatment of Presumptive Zygotes

The effects of retinol and retinoic acid on early embryonic development of in vitro produced bovine embryos were investigated. Oocytes and their surrounding cumulus cells were collected from bovine ovaries, matured in vitro for 24 h and fertilized by standard procedures for 8-10 h. Presumptive zygotes were denuded of cumulus cells, washed and cultured in modified synthetic oviduct fluid (a standard culture medium) in the presence or absence of retinol (1.0 or 10.0 mM) at 38.5° C. in a humidified atmosphere of 5% CO₂ in air. Embryos were cultured for 7-8 days.

Blastocyst (a fluid filled ball of approximately 100 cells, containing a inner cell mass which gives rise to the fetus and a trophoblast which gives rise to the placenta) formation was used as an end point for in vitro development. The above experiment was performed three times using 210 embryos in each treatment group with 70 embryos/group (630 total, 210/treatment). Embryonic development to the blastocyst stage, expressed as % blastocyst/cleaved embryos, was control (no retinol)=28.33%, 1 uM Retinol=48.66%, 10 uM Retinol=57.33%. Retinol significantly (p<0.01) increased development to the blastocyst stage in all experiments. Similar positive results were observed when the culture medium contained 1 uM RA.

### EXAMPLE 3

### In Vitro Retinol Treatment of Isolated Oocytes

As experiments performed in sheep demonstrated that retinol administration during follicular development significantly improved the in vitro viability of resultant embryos following fertilization, in vitro exposure of oocytes to retinoids, prior to in vitro fertilization, is specifically envisioned to result in improved developmental competence of in vitro produced embryos. In this example, oocytes are harvested from the ovaries of animals by ultrasound guided aspiration and/or slicing of follicles from ovaries collected at a slaughter facility. The isolated oocytes are washed several times in a buffered physiological salt solution and transferred to an appropriate in vitro maturation medium such as tissue culture medium 199 (TCM-199, Sigma Chemical Co.) containing 0.2 mM pyruvate, 5.0 ug/ml FSH, 1.0 ug/ml estradiol and 10% fetal bovine serum (Sirard MA, Parrish JJ, Ware, CB Leibfried-Rutledge, ML and First NL, Biology of Reproduction, 1988;39:546-552). A retinoid, such as retinol or retinoic acid, is included in the maturation medium at a concentration of 0.001-100.0 micromolar (the most effective concentrations can be determined empirically using the methods described herein). Oocytes are then cultured for a period of 20-48 h at 37-39 degrees C. in an atmosphere of 5% CO2 and air. Following incubation of oocytes in medium containing retinoid, oocytes are washed, fertilized, and then implanted in a uterus or fallopian tube.

### EXAMPLE 4

### Retinoid Treatment Enhances Pregnancy Rates

Twenty-four ewes were synchronized, superovulated and administered treatments, as described previously (Eberhardt, Will and Godkin. Biol Reprod 60, 1483-1487, 1999). Briefly, animals received a progestagen implant and six days later received two injections of prostaglandin F2-alpha (Lutalyse, Upjohn Co. Kalamazoo, Mich.) twelve hours apart. Superovulation was induced with 24 units of porcine follicle stimulating hormone (FSH, Sioux Biochemical, Sioux City, Iowa) in decreasing doses over 3 days beginning 9-11 days after implant administration. All-trans retinol was administered to twelve ewes in corn oil at 500,000 IU per injection on the first and last day of FSH injections. Control ewes (12) received corn oil without retinol. Implants were removed at the time of the fifth FSH injection and animal exhibited estrous behavior 24-34 hours later and were bred to intact rams.

Embryos at the morula stage of development (168 h post implant removal) were collected by gently flushing the uterus with phosphaste-buffered saline+2% bovine serum albumin (BSA) following surgical hysterectomy. Embryos were frozen by conventional methods (Leibo. Theriogenology 21: 767. 1984) using ethylene glycol as the cryoprotectant in a programmable freezer and stored in liquid N₂. After three months, embryos were thawed briefly (30 seconds) at 37° C. and two embryos were transferred directly into the uterine horn of synchronized recipient ewes. Seven recipient ewes received embryos from retinol-treated ewes and 11 recipient ewes received embryos from control ewes. Pregnancy rates were determined by ultrasonography 30-40 days after transfer.

Six of the seven ewes (86%) receiving embryos from retinol-treated ewes were determined to be pregnant, whilst five of eleven ewes (45%) receiving embryos from control treated ewes were observed to be pregnant. Of the six pregnant ewes carrying embryos from retinol-treated ewes, five were observed to be carrying twins and one was carrying a single embryo. All five of the pregnant ewes carrying embryos from control ewes were observed to be carrying single embryos. Of all transferred embryos from retinol-treated ewes, 78.6% survived, versus a 22.7% survival rate of embryos from control-treated ewes. Results clearly demonstrate that our retinol-treatment protocol improves embryonic survival.

Other Embodiments

While the above specification contains many specifics, these should not be construed as limitations on the scope of the invention, but rather as examples of preferred embodiments thereof. Many other variations are possible. For example, it is specifically envisioned that retinoids can be added to ova isolated from a human female or presumptive zygotes during standard IVF procedures to enhance survival of embryos. One or more of these retinoid-treated embryos can then be implanted into the female or another female to enhance the chance that the embryo(s) will survive.

What is claimed is:

1. A method for enhancing reproductive success in an animal comprising the step of administering to the animal (a) a preparation comprising a retinoid in an amount effective to enhance the reproductive success of the animal and (b) an agent that stimulates superovulation in an amount sufficient to stimulate superovulation in the animal.

2. The method of claim 1, wherein the preparation further comprises a pharmaceutically acceptable carrier.

3. The method of claim 1, wherein the step of administering the preparation is performed by a parenteral route.

4. The method of claim 4, wherein the parenteral route is by injection.

5. The method of claim 1, wherein the retinoid is all-trans retinol.

6. The method of claim 5, wherein the all-trans retinol is administered to the animal in a dosage of 500 to 50,000 IU/Kg.

7. The method of claim 6, wherein the all-trans retinol is administered to the animal in a dosage of 1000 to 25,000 IU/Kg.

8. A method for enhancing the viability of an embryo comprising the steps of:
(a) isolating an ovum from an animal;
(b) fertilizing the isolated ovum to form an embryo; and
(c) exposing the embryo to a purified retinoid.

9. The method of claim 8, wherein the embryo is exposed to the purified retinoid at a concentration of about 0.5 to 50 micromolar.

10. The method of claim 9, wherein the embryo is exposed to the purified retinoid for a period of at least one hour.

11. The method of claim 8 further comprising the step (d) of implanting the embryo in a uterus or a fallopian tube.

12. The method of claim 8, wherein the retinoid is retinol.

13. The method of claim 8, wherein the retinoid is retinoic acid.

14. An embryo made according to a process comprising the steps of:
(a) isolating an ovum from an animal;
(b) fertilizing the isolated ovum to form an embryo; and
(c) exposing the embryo to a purified retinoid.

15. The embryo of claim 14, wherein the retinoid is retinol.

16. The embryo of claim 14, wherein the retinoid is retinoic acid.

17. A method for enhancing the viability of an embryo comprising the steps of:
(a) isolating an ovum from an animal;
(b) exposing the isolated ovum to a purified retinoid; and
(c) fertilizing the ovum to form an embryo.

18. The method of claim 17, wherein the isolated ovum is exposed to the purified retinoid at a concentration of about 0.5 to 50 micromolar.

19. The method of claim 17, wherein the isolated ovum is exposed to the purified retinoid for a period of at least one hour.

20. The method of claim 17 further comprising the step (d) of implanting the embryo in a uterus or a fallopian tube.

22. An embryo made according to a process comprising the steps of:
(a) isolating an ovum from an animal;
(b) exposing the isolated ovum to a retinoid; and
(c) fertilizing the ovum to form an embryo.

23. A kit for enhancing reproductive success in an animal, the kit comprising at least one dose of a purified retinoid, and written instructions for administering the at least one dose to the animal, the at least one dose comprising a sufficient amount of the retinoid to enhance the reproductive success of the animal after being administered to the animal

24. The kit of claim 23, wherein the retinoid is retinol.

25. The kit of claim 23, wherein the at least one dose of a retinoid is formulated for parenteral administration.

26. The kit of claim 25, wherein the retinoid is mixed with a pharmaceutically acceptable carrier.
